(19) United States
(12) Patent Application Publication
(10) Pub. No.: US 2024/0148458 A1
(43) Pub. Date: May 9, 2024

(54) SYSTEMS AND METHODS FOR COUPLING AN INSTRUMENT TO AN INSTRUMENT DRIVE SYSTEM

(71) Applicant: INTUITIVE SURGICAL OPERATIONS, INC., Sunnyvale, CA (US)

(72) Inventor: David W. Bailey, Portola Valley, CA (US)

(21) Appl. No.: 18/501,430

(22) Filed: Nov. 3, 2023

Related U.S. Application Data
(60) Provisional application No. 63/422,827, filed on Nov. 4, 2022.

(51) Int. Cl. A61B 34/00

(52) U.S. Cl. CPC A61B 34/73; A61B 34/25; A61B 2034/2061

(57) ABSTRACT

A system comprises an instrument drive system that comprises a first housing, a first magnetic coupling member within the first housing, and a motor mechanically coupled to the first magnetic coupling member. The motor is configured to rotate the first magnetic coupling member. The system further comprises a medical instrument that comprises a second housing, a rotor within the second housing, and a second magnetic coupling member within the second housing and mechanically coupled to the rotor. The second magnetic coupling member is spaced from and magnetically coupled to the first magnetic coupling member. Rotation of the first magnetic coupling member causes rotation of the second magnetic coupling member and the rotor.

### CROSS-REFERENCED APPLICATIONS

This application claims priority to and the benefit of U.S. Provisional Application No. 63/422,827, filed Nov. 4, 2022, and entitled “Systems and Methods for Coupling an Instrument to an Instrument Drive System,” which is incorporated by reference herein in its entirety.

### FIELD

Examples described herein relate to systems and methods for magnetically coupling an instrument to an instrument drive system.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, an operator may insert minimally invasive medical tools to reach a target tissue location. Minimally invasive medical tools include instruments such as therapeutic, diagnostic, biopsy, and surgical instruments. Minimally invasive medical tools may be coupled to drive systems that drive the minimally invasive medical tools.

### SUMMARY

Various features may improve the connection between an instrument drive system and an instrument (e.g., a medical instrument) while allowing for sealing between the instrument and the instrument drive system during operation. The following presents a simplified summary of various examples described herein and is not intended to identify key or critical elements or to delineate the scope of the claims.

Consistent with some examples, a system is provided. The system includes an instrument drive system that includes a first housing, a first magnetic coupling member within the first housing, and a motor mechanically coupled to the first magnetic coupling member. The motor is configured to rotate the first magnetic coupling member. The system further includes a medical instrument. The medical instrument includes a second housing, a rotor within the second housing, and a second magnetic coupling member within the second housing and mechanically coupled to the rotor. The second magnetic coupling member is spaced from and magnetically coupled to the first magnetic coupling member. Rotation of the first magnetic coupling member causes rotation of the second magnetic coupling member and the rotor.

Consistent with other examples, a system is provided. The system includes an instrument drive system that includes a first magnetic coupling member and a motor mechanically coupled to the first magnetic coupling member. The motor is configured to rotate the first magnetic coupling member. The system further includes a medical instrument. The medical instrument includes a rotor and a second magnetic coupling member mechanically coupled to the rotor. The second magnetic coupling member is spaced from and magnetically coupled to the first magnetic coupling member. The medical instrument further includes a flexible elongate device. Rotation of the first magnetic coupling member causes rotation of the second magnetic coupling member and the rotor, and rotation of the rotor controls articulation of the flexible elongate device.

Consistent with other examples, a system for coupling a medical instrument to an instrument drive system is provided. The system includes a first magnetic coupling member positioned within a housing of the instrument drive system. The first magnetic coupling member includes a first position sensor. The system further includes a second magnetic coupling member spaced from and magnetically coupled to the first magnetic coupling member. The second magnetic coupling member includes a second position sensor. The second magnetic coupling member is mechanically coupled to the medical instrument and is positioned within a housing of the medical instrument. Rotation of the first magnetic coupling member causes corresponding rotation of the second magnetic coupling member, and rotation of the second magnetic coupling member causes corresponding rotation of a rotor of the medical instrument. The system further includes a control system that includes a processor and a memory. The memory includes machine readable instructions that, when executed by the processor, cause the control system to: receive position data from the first and second position sensors indicating a position of the first position sensor relative to the second position sensor; and based on the received position data, determine a torque applied to the second magnetic coupling member.

Other examples include corresponding computer systems, apparatus, and computer programs recorded on one or more computer storage devices, each configured to perform the actions of any one or more methods described below.

It is to be understood that both the foregoing general description and the following detailed description are illustrative and explanatory in nature and are intended to provide an understanding of the various examples described herein without limiting the scope of the various examples described herein. In that regard, additional aspects, features, and advantages of the various examples described herein will be apparent to one skilled in the art from the following detailed description.

### BRIEF DESCRIPTIONS OF THE DRAWINGS

FIG. 1A illustrates an instrument manipulation system including an instrument drive system and an instrument according to some examples.

FIG. 1B illustrates a backend mechanism of an instrument in a partially exploded configuration according to some examples.

FIG. 2A illustrates a cross-sectional view of an instrument coupled to an instrument drive system according to some examples.

FIG. 2B illustrates a cross-sectional view of an instrument coupled to an instrument drive system according to some examples.

FIGS. 3A and 3B illustrates a magnetic coupling member of an instrument coupled to a magnetic coupling member of an instrument drive system according to some examples.

FIG. 3C illustrates a magnetic field when a magnetic coupling member of an instrument is coupled to a magnetic coupling member of an instrument drive system according to some examples.

FIG. 4 illustrates a schematic top view of a housing of an instrument drive system with multiple magnetic coupling members according to some examples.

FIG. 5 is a flowchart illustrating a method of determining a torque applied to a magnetic coupling member of an instrument according to some examples.

FIG. 6 is a simplified diagram of a computer-assisted, teleoperated system according to some examples.

FIG. 7 is a simplified diagram of a medical instrument system according to some examples.

Various examples described herein and their advantages are described in the detailed description that follows. It should be appreciated that like reference numerals are used to identify like elements illustrated in one or more of the figures for purposes of illustrating but not limiting the various examples described herein.

### DETAILED DESCRIPTION

In the following description, specific details describe some examples consistent with the present disclosure. Numerous specific details are set forth in order to provide a thorough understanding of the examples. It will be apparent to one skilled in the art, however, that some examples may be practiced without some or all of these specific details. The specific examples disclosed herein are meant to be illustrative but not limiting. One skilled in the art may realize other elements that, although not specifically described, are within the scope and the spirit of this disclosure. In addition, to avoid unnecessary repetition, one or more features shown and described in association with one example may be incorporated into other examples unless specifically described otherwise or if the one or more features would make an example non-functional. In some instances, well known methods, procedures, components, and circuits have not been described in detail so as not to unnecessarily obscure aspects of the examples.

An instrument may be coupled to an instrument drive system before and/or during a medical procedure. The instrument drive system controls the movement of the instrument. In one example, the instrument is a flexible catheter with an articulable distal tip. The instrument drive system controls the articulation of the distal tip, such as along pitch and yaw axes. The instrument may be magnetically coupled to the instrument drive system, controlling movement of the instrument via electromagnetic force rather than mechanical force. Magnetically coupling the instrument and the instrument drive system may obviate the need for mechanical connections, allowing for sealing between the instrument and the instrument drive system during operation. For example, a seal may be placed around the instrument and/or the instrument drive system, and the instrument and the instrument drive system may be coupled without the need for a torque coupling through a sterile barrier, e.g., a sterile adapter. The sealed instrument and instrument drive system may have greater reliability than unsealed instruments and instrument drive systems. For example, cleaning fluids or other fluids and/or debris may be prevented from entering the sealed instrument and the sealed instrument drive system. The magnetic coupling between the instrument and the instrument drive system also provides various other advantages over mechanical coupling. For example, the magnetic coupling accommodates misalignment between rotational axes of the instrument and the instrument drive system so that the instrument drive system may still drive the instrument even if the axes are misaligned. The accommodation of the misalignment may be achieved without the need for an additional coupling mechanism, e.g., an Oldham coupling.

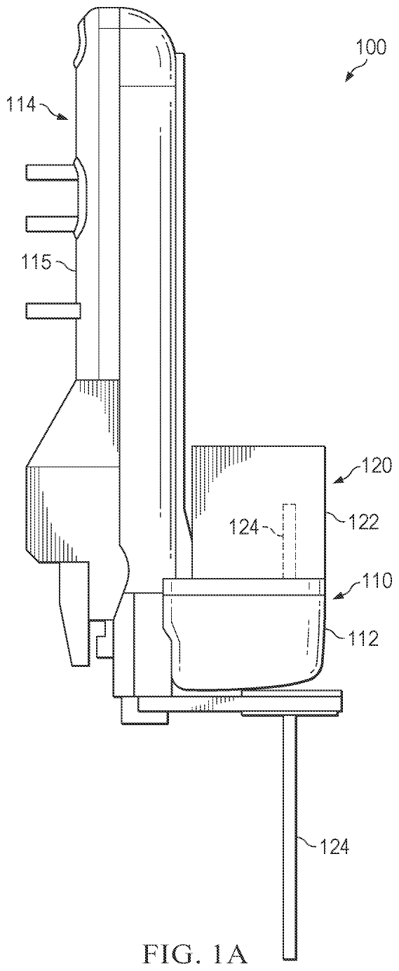

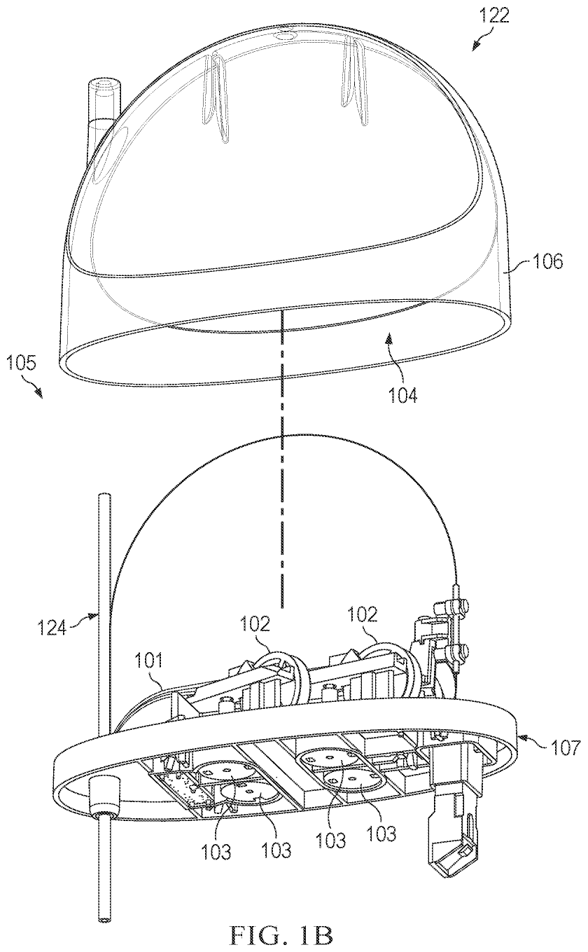

FIG. 1A illustrates an instrument manipulation system 100 including an instrument drive system 110 and an instrument 120 (e.g., a medical instrument). The instrument drive system 110 includes a housing 112 and an instrument holder 114. The instrument holder 114 includes an instrument holder frame 115. In some examples, the housing 112 of the instrument drive system 110 is coupled to the medical instrument 120, which may be an elongate instrument. The medical instrument 120 includes a housing 122 and an elongate body 124, which may be an elongate, flexible body of a flexible elongate device. The housing 112 may move in a distal and proximal direction relative to the instrument holder frame 115. This motion may also move the instrument housing 122 and the elongate body 124 in the distal and proximal direction relative to the instrument holder frame 115.

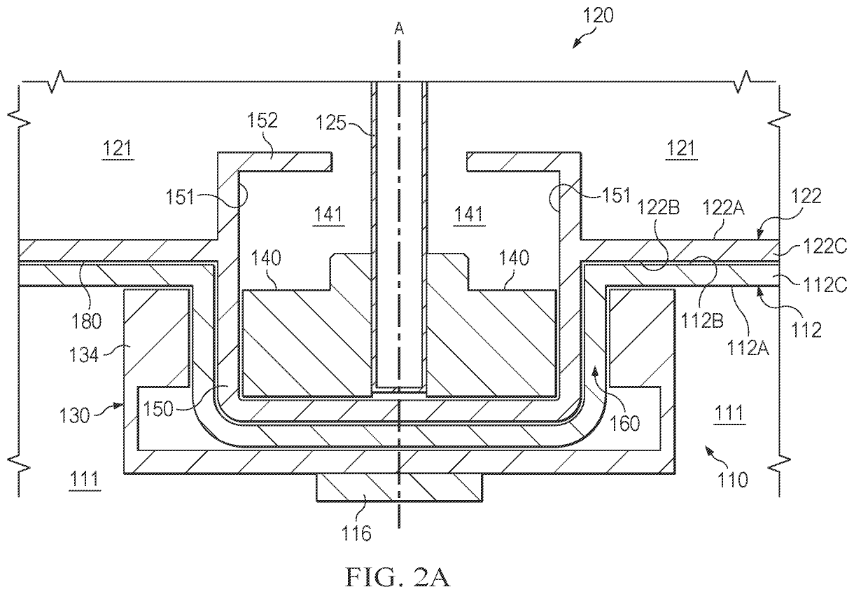

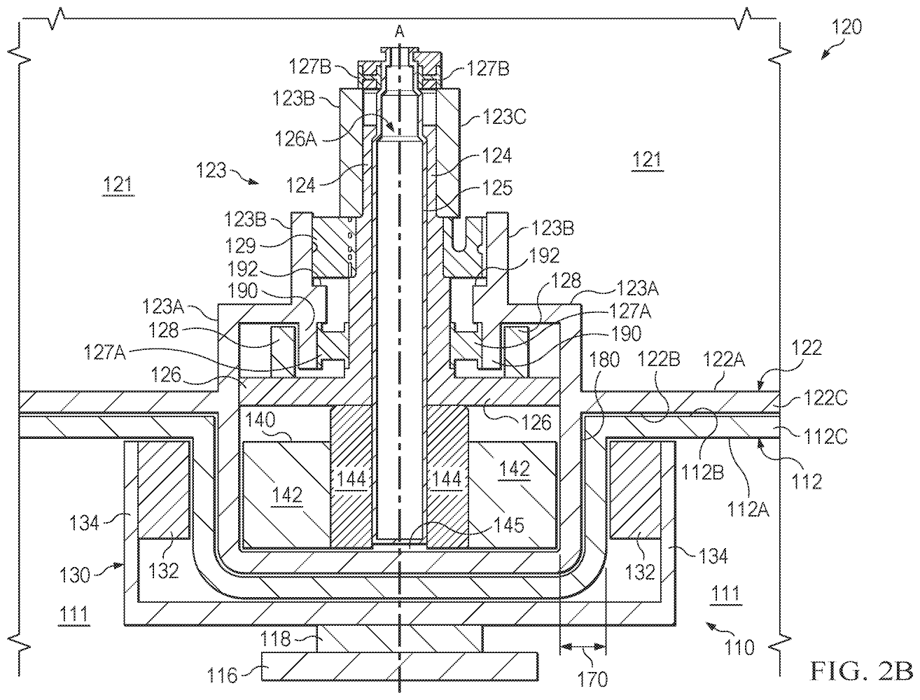

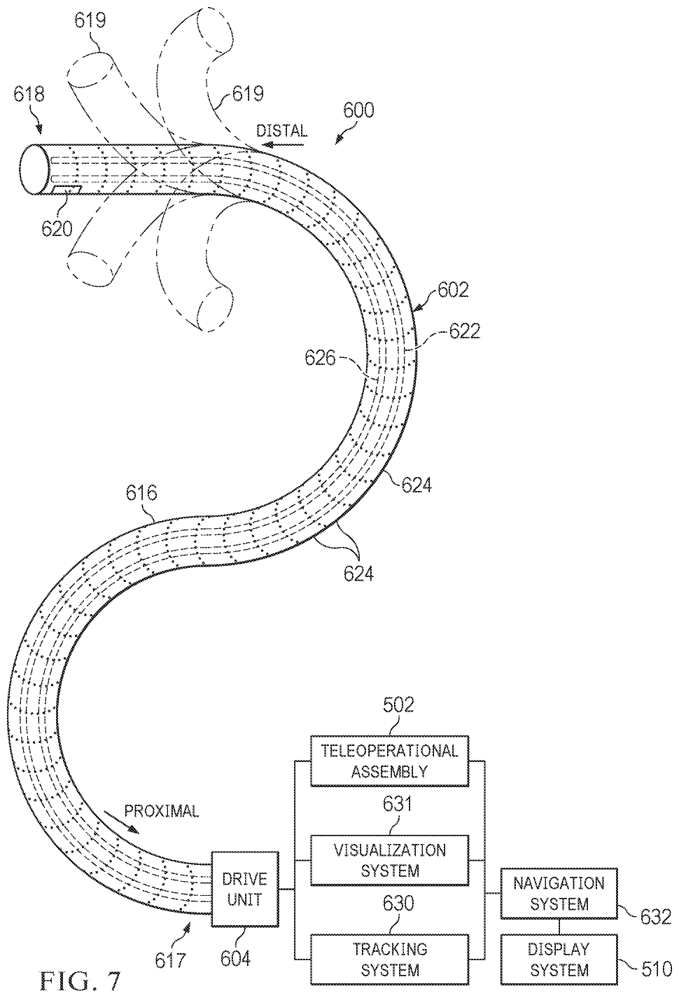

FIG. 1B illustrates a backend mechanism 105 of an instrument (e.g., the medical instrument 120) in a partially exploded configuration. The backend mechanism 105 includes the housing 122. The housing 122 includes a cover 106 and a chassis 107. The elongate body 124 extends through and terminates within the housing 122. The backend mechanism also includes a plurality of pull wires 101, which may extend from the elongate body 124 to one or more steering components 102. The steering components 102 are arranged to direct the pull wires 101 that extend from the elongate body 124 to drive components 103. The drive components 103 may interact with corresponding drive components of the instrument drive system 110 to articulate the elongate body 124 of the medical instrument 120 via control of the pull wires 101. For example, the pull wires 101 may be axially tightened or loosened to displace the distal end of the elongate body 124, as described below with respect to FIG. 7. In some examples, the drive components 103 of the medical instrument 120 may be magnetically coupled to the corresponding drive components of the instrument drive system 110. The interaction between the drive components 103 and the drive components of the instrument drive system 110 will be discussed in greater detail below with respect to FIGS. 2A and 2B.

The cover 106 may include a cavity 104 sized and arranged to cover and protect the steering components 102, drive components 103, and other components carried by the chassis 107. The housing 122 of the medical instrument 120 is selectively attachable to the housing 112 of the instrument drive system 110 and provides a compact manageable unit that securely protects steering and sensing components from an exterior environment outside of the housing 122. In some examples, the housing 122 is magnetically coupled to the housing 112. In some examples, the housing 122 is mechanically coupled to the housing 112.

FIGS. 2A and 2B illustrate a cross-sectional view at an interface for magnetic coupling between the instrument drive system 110 and the medical instrument 120. The instrument manipulation system 100 may include more than one magnetic coupling. For example, the instrument drive system 110 may include one magnetic coupling for each pull wire of the medical instrument 120. The instrument drive system 110 may be coupled to the medical instrument 120 to drive movement of the medical instrument 120, such as the articulation of the medical instrument 120. In some examples, the instrument drive system 110 may be magnetically coupled to the medical instrument 120 to drive movement of the medical instrument 120 using electromagnetic force. As shown in FIG. 2A, the instrument drive system 110 may include a housing 112, a motor 116, and a magnetic coupling member 130. The magnetic coupling member 130 and the motor 116 may be positioned within the housing 112 of the instrument drive system 110. The motor 116 may be mechanically coupled to the magnetic coupling member 130 such that rotation of the motor 116 causes rotation of the magnetic coupling member 130. The motor 116 may be coupled to the magnetic coupling member 130 in different ways, such as a press fit, a pin connection, a keyed connection, an adhesive connection, or with the use of any other mechanical coupling. The medical instrument 120 may include a housing 122, a rotor 125, and a magnetic coupling member 140. The rotor 125 and the magnetic coupling member 140 are positioned within the housing 122 of the medical instrument 120. The rotor 125 and the magnetic coupling member 140 may be mechanically coupled such that rotation of the magnetic coupling member 140 causes rotation of the rotor 125. The magnetic coupling member 140 may be coupled to the rotor 125 in different ways, such as a press fit, a pin connection, a keyed connection, an adhesive connection, or with the use of any other mechanical coupling. In some examples, the rotor 125 may extend within an aperture (e.g., a central channel) of the magnetic coupling member 140. The rotor 125 may be axially aligned with the magnetic coupling member 140 about an axis A. The axis A may be a longitudinal axis of the rotor 125 and the rotor 125 may rotate along the axis A. The magnetic coupling member 140 may be spaced apart from and magnetically coupled to the magnetic coupling member 130.

In operation, power is applied to the motor 116 causing the motor 116 rotate, and thus also causing the mechanically coupled magnetic coupling member 130 to correspondingly rotate. Because the magnetic coupling members 130, 140 are magnetically coupled, when the magnetic coupling member 130 rotates, the magnetic coupling member 130 causes the magnetic coupling member 140 to correspondingly rotate via the magnetic coupling. The rotation of the magnetic coupling member 140 causes the mechanically coupled rotor 125 to correspondingly rotate. Therefore, rotation of the motor 116 causes corresponding rotation of the rotor 125. In this way, the instrument drive system 110 may drive movement of the medical instrument 120.

The housing 112 of the instrument drive system 110 includes an interior surface 112A, an exterior surface 112B, and a wall 112C extending between the interior surface 112A and the exterior surface 112B. The interior surface 112A defines an interior 111 of the housing 112. The housing 122 of the medical instrument 120 includes an interior surface 122A, an exterior surface 122B, and a wall 122C extending between the interior surface 122A and the exterior surface 122B. The interior surface 122A defines an interior 121 of the housing 122. The housing 122 of the medical instrument 120 may further include a protruding portion 150 and an interior portion 152. The protruding portion 150 and interior portion 152 define an interior surface 151 that encloses a cavity 141. The magnetic coupling member 140 may be positioned within the cavity 141. In some examples, the cavity 141 prevents the magnetic coupling member 140 from axially migrating along the axis A of the rotor 125.

The housing 112 of the instrument drive system 110 may further include a recessed portion 160. In some examples, the protruding portion 150 of the housing 122 may be positioned within (e.g., nested within) the recessed portion 160 of the housing 112. The magnetic coupling member 130 may be positioned around the interior surface 112A of the recessed portion 160 of the housing 112. The magnetic coupling member 140 may be positioned within the cavity 141 of the protruding portion 150 of the housing 122. Therefore, in some examples, the magnetic coupling member 140 may be positioned within (e.g., nested within) the magnetic coupling member 130 as a result of the protruding portion 150 being positioned within (e.g., nested within) the recessed portion 160. In some examples, the magnetic coupling member 130 may have a toroidal shape, such as a donut, a ring, or any other toroid. In some examples, the magnetic coupling member 140 may have a toroidal shape, such as a donut, a ring, or any other toroid.

In some examples, the nesting of the protruding portion 150 and the recessed portion 160 may be reversed. For example, the housing 122 of the medical instrument 120 may include the recessed portion 160, and the housing 112 of the instrument drive system 110 may include the protruding portion 150. In such examples, the protruding portion 150 of the housing 112 may be positioned within (e.g., nested within) the recessed portion 160 of the housing 122. The magnetic coupling member 140 may be positioned around an interior surface of the recessed portion 160. The magnetic coupling member 130 may be positioned within the cavity 141 of the protruding portion 150. Therefore, in some examples, the magnetic coupling member 130 may be positioned within (e.g., nested within) the magnetic coupling member 140 as a result of the protruding portion 150 being positioned within (e.g., nested within) the recessed portion 160.

The instrument drive system 110 may be coupled to the medical instrument 120 via the magnetic coupling members 130, 140. For example, the magnetic coupling member 130 of the instrument drive system 110 is magnetically coupled to the magnetic coupling member 140 of the medical instrument 120. The magnetic coupling of the members 130, 140 may magnetically couple the instrument drive system 110 and the medical instrument 120 to each other. The magnetic coupling between the instrument drive system 110 and the medical instrument 120 may allow for the instrument drive system 110 and the medical instrument 120 to be securely sealed. Because the instrument drive system 110 and the medical instrument 120 may be magnetically coupled, the housing 112 and the housing 122 may, alone or in combination, form a fixed seal that prevents ingress, egress, and/or migration of fluid and debris into the instrument drive system 110, into the medical instrument 120, and/or between the instrument drive system 110 and the medical instrument 120. For example, the wall 112C of the housing 112 may be a solid wall, which may prevent fluid from entering the interior 111 of the housing 112. The solid wall 112C may also prevent debris, such as particles of the patient anatomy, particles of the operator's anatomy (e.g., hair, skin, etc.) particles of medical equipment, clothing particles, or any other solid particles, from entering the interior 111 of the housing 112. The wall 122C of the housing 122 may also be a solid wall, which may prevent fluid and debris from entering the interior 121 of the housing 122. In some examples, the wall 122C of the housing 122 may prevent fluid from entering the interior 121 of the housing 122 when the housing 122 is being cleaned and/or sterilized. In some examples, the walls 112C, 122C may each be welded and/or glued to form a fixed seal.

In some examples, the wall 112C of the housing 112 and the wall 122C of the housing 122 may be made of non-magnetic material to reduce the affect the walls 112C, 122C may have on the magnetic force coupling the magnetic coupling members 130, 140. In some examples, the walls 112C, 122C may also be non-conductive. The material(s) used for the housings 112, 122 may be chemically compatible with the cleaning agents used to clean the housings 112, 122 to prevent the cleaning agents from degrading the housings 112, 122 over time.

In some examples, a sterile drape may be used before and/or during a medical procedure. The sterile drape may be placed in a gap 180 between the housings 112, 122 without interfering with the magnetic coupling between the medical instrument 120 and the instrument drive system 110.

As shown in FIG. 2B, in some examples, the instrument drive system 110 and the medical instrument 120 may include additional features. For example, the instrument drive system 110 may further include a gear box 118. The gear box 118 may be mechanically coupled to the motor 116 and to the magnetic coupling member 130. For example, a rotor of the motor 116 may be coupled to the gear box 118. The gear box 118 is configured to engage the magnetic coupling member 130 such that rotation of the motor 116 may cause rotation of the gear(s) within the gear box 118, which may in turn cause rotation of the magnetic coupling member 130. For example, the rotor of the motor 116 may provide torque to one or more gears of the gear box 118, which may provide torque to the magnetic coupling member 130. In some examples, the torque provided by the rotor of the motor 116 to the gear(s) of the gear box 118 may be less than the torque provided by the gear(s) to the magnetic coupling member 130. Alternatively, the torque provided by the rotor of the motor 116 to the gear(s) of the gear box 118 may be greater than the torque provided by the gear(s) to the magnetic coupling member 130. Any one or more of the gear box 118, the motor 116, or the magnetic coupling member 130 may be positioned within the interior 111 of the housing 112.

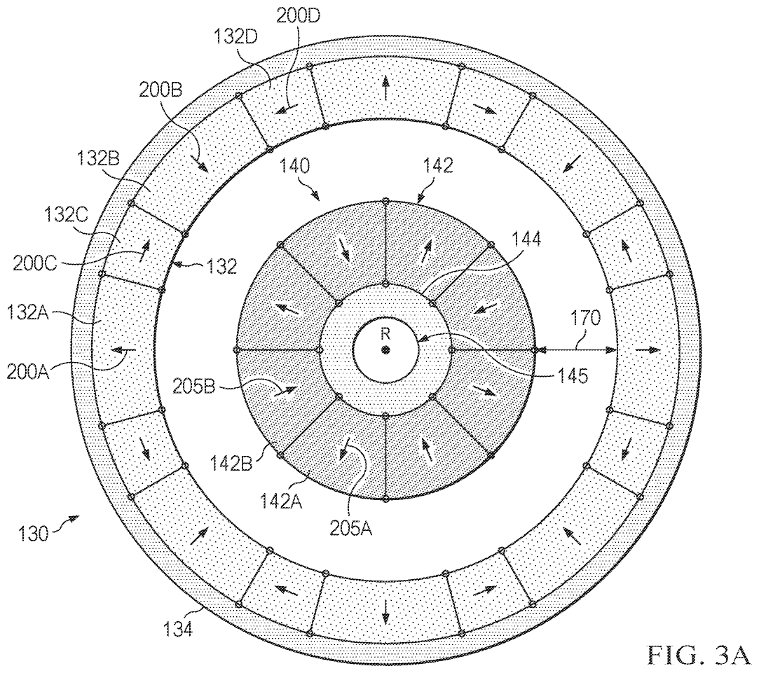

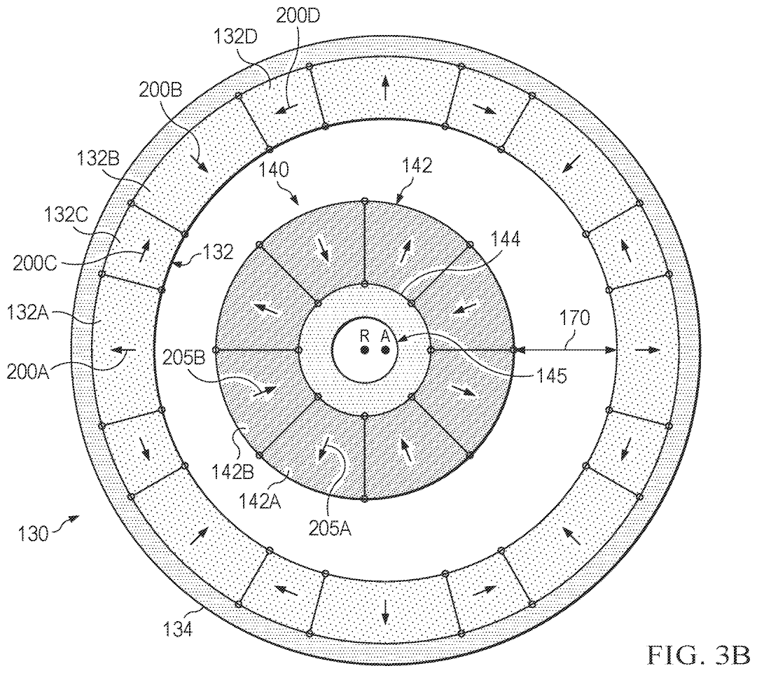

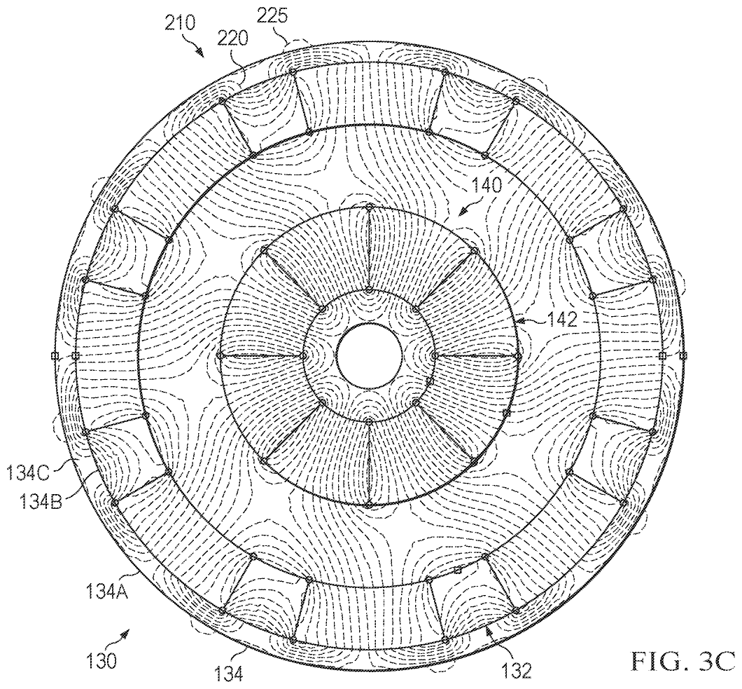

In some examples, the magnetic coupling member 130 may include a magnet array 132 and a housing 134. The magnet array 132 may include one or more individual magnets, which may be arranged in a variety of configurations with varying directions of polarity for each magnet in the magnet array 132. Additional details regarding the magnet array 132 are provided below with respect to FIGS. 3A-3C. In some examples, each magnet in the magnet array 132 is coupled to the housing 134. The magnets in the magnet array 132 may be coupled to the housing 134 in any number of ways, such as a press fit, a pin connection, a keyed connection, an adhesive connection, or with the use of any other mechanical coupling. In some examples, the housing 134 may be steel, iron, or any other magnetic material.

In some examples, the motor 116 is coupled (e.g., releasably coupled or fixedly coupled) to the magnetic coupling member 130. As shown in FIG. 2B, the motor 116 may be releasably coupled or fixedly coupled to the gear box 118, which may be releasably coupled or fixedly coupled to the magnetic coupling member 130 (e.g., to the housing 134). Alternatively, the motor 116 may be releasably coupled or fixedly coupled to the magnetic coupling member 130 directly, as shown in FIG. 2A. For example, the motor 116 may be directly coupled (e.g., releasably coupled or fixedly coupled) to the housing 134 of the magnetic coupling member 130. As discussed above, the motor 116 may be coupled to the magnetic coupling member 130, either directly or via the gear box 118, such that the magnetic coupling member 130 rotates with the motor 116.

As shown in FIG. 2B, the medical instrument 120 may further include one or more casing portions 123, a shaft 126, a proximal bearing 127A, a distal bearing 127B, a shuttle 128, and a pulley 129. Any one or more of the rotor 125, the shaft 126, the proximal bearing 127A, the pulley 129, or the magnetic coupling member 140 may be positioned within the cavity 141 of the housing 122.

In some examples, the magnetic coupling member 140 may include a magnet array 142 and an inner shaft 144. The magnet array 142 may include one or more individual magnets, which may be arranged in a variety of configurations with varying directions of polarity for each magnet in the magnet array 142. Additional details regarding the magnet array 142 are provided below. In some examples, each magnet in the magnet array 142 is coupled to the inner shaft 144. The magnets in the magnet array 142 may be coupled to the inner shaft 144 in any number of ways, such as a press fit, a pin connection, a keyed connection, an adhesive connection, or with the use of any other mechanical coupling. In some examples, the inner shaft 144 may be steel, iron, or any other magnetic material.

In some examples, the magnetic coupling member 140 may be coupled to the rotor 125. As shown in FIG. 2B, the inner shaft 144 of the magnetic coupling member 140 may be coupled to the rotor 125. In some examples, the rotor 125 extends within a channel 145 defined by the inner shaft 144. The rotor 125 may be coupled to the inner shaft 144 in any number of ways, such as a press fit, a pin connection, a keyed connection, an adhesive connection, a welded connection, or with the use of any other mechanical coupling. Due to the connection between the inner shaft 144 of the magnetic coupling member 140 and the rotor 125, rotation of the magnetic coupling member 140 causes corresponding rotation of the rotor 125. The rotor 125 rotates about its longitudinal axis A. Further details regarding the interaction between the magnetic coupling member 140 and the rotor 125 will be discussed below.

As further shown in FIG. 2B, the rotor 125 may extend within a channel 126A defined by the shaft 126. The rotor 125 may be coupled to the shaft 126 in any number of ways, such as a press fit, a pin connection, a keyed connection, an adhesive connection, a welded connection, or with the use of any other mechanical coupling. In some examples, due to the connection between the rotor 125 and the shaft 126, rotation of the rotor 125 causes corresponding rotation of the shaft 126. In some examples, rotation of the rotor 125 causes movement of the flexible body 124. For example, rotation of the rotor 125 may control articulation of a distal portion of the elongate, flexible body 124, which may include the rotor 125 and the shaft 126. Various types of movements may also be controlled via the rotation of the rotor 125.

As shown in FIG. 2B, the housing 122 may further include three casing portions 123A, 123B, 123C. However, the housing 122 may include any number of casing portions, such as one casing portion, two casing portions, four casing portions, or more casing portions. In some examples, the casing portions 123 may have different diameters. For example, the casing portion 123A has a bigger diameter than the casing portion 123B, which has a bigger diameter than the casing portion 123C. In some examples, the casing portions 123 may have the same diameters. In some examples, at least two of the casing portions 123 may have the same diameter, which may be a different diameter than other casing portions. Any other configuration of casing portions may be used. The casing portions 123 may provide reinforcement for the rotor 125 and may align the rotor 125 with the other components that may be housed within the casing portions 123, such as the shaft 126, the proximal bearing 127A, the distal bearing 127B, the shuttle 128, and/or the pulley 129.

The casing portion 123A may house the shaft 126, the proximal bearing 127A, and the shuttle 128. The casing portion 123A may include a projection 190 that separates the proximal bearing 127A and the shuttle 128. This separation may reduce any interference between the proximal bearing 127A and the shuttle 128. In some examples, the shaft 126 rotates within the casing portion 123A. The shaft 126 may rotate about the longitudinal axis A of the rotor 125. A portion of the shaft 126 may rotate within the proximal bearing 127A. The shuttle 128 may stop rotation of the shaft 126 when the shaft 126 reaches a rotation threshold. For example, the rotation threshold may be 360°, or one full rotation of the shaft 126. If the shaft 126 rotates 360°, the shuttle 128 will prevent any further rotation of the shaft 126. The rotation threshold may be set at any other rotation amount and may be customized for the particular instrument used in the instrument manipulation system 100. For example, the rotation threshold may be set at 270° (three quarters of one full rotation of the shaft 126), 180° (one half of one full rotation of the shaft 126), or any other amount of rotation.

In some examples, the casing portion 123B may house the pulley 129, as shown in FIG. 2B. The casing portion 123B may include a ledge 192 that may provide clearance for the pulley 129 as the pulley 129 rotates. In some examples, the elongate body 124 includes a cable (not shown), such as a control cable, that may be used to drive and/or steer the elongate body 124. The cable may be referred to as a pull wire. The pull wire may be routed around the pulley 129, which may be referred to as a pull wire pulley. In some examples, rotation of the drive element causes a change in tension of the pull wire, resulting in steering of the elongate body 124.

In some examples, the casing portion 123C may house a portion of the shaft 126, as shown in FIG. 2B. The portion of the shaft 126 within the casing portion 123C is a more distal portion than the portion of the shaft 126 within the casing portion 123A. FIG. 2B shows the distal bearing 127B positioned outside of and distal to the casing portion 123C. In some examples, the distal bearing 127B may be positioned within the casing portion 123C.

In some examples, the magnetic coupling members 130, 140 together extends across a gap 170. The gap 170 may be an air gap but may also be filled with any other suitable fluid. The gap 170 is the distance between the magnet array 132 of the magnetic coupling member 130 and the magnet array 142 of the magnetic coupling member 140. The gap 170 extends across the walls 112C, 122C. In examples where other configurations of magnetic coupling members may be used, the gap 170 would extend across any components or structures that may be between the magnet arrays of the magnetic coupling members. In some examples, a sterile barrier extends within the gap 170 between the housings 112, 122. For example, a sterile barrier may extend within the gap 180 between the housings 112, 122.

As discussed above, rotation of the motor 116 may cause the magnetic coupling member 130 of the instrument drive system 110 to rotate. In some examples, the magnetic coupling member 130 may rotate about the longitudinal axis A of the rotor 125. Due to the magnetic coupling between the magnetic coupling member 130 and the magnetic coupling member 140 of the medical instrument, rotation of the magnetic coupling member 130 causes corresponding rotation of the magnetic coupling member 140. For example, rotation of the magnet array 132 (which may be a driving magnet) may cause the magnet array 142 (which may be a driven magnet) to correspondingly rotate. As discussed above, rotation of the magnetic coupling member 140 causes corresponding rotation of the rotor 125. As further discussed above, rotation of the rotor 125 causes corresponding rotation of the shaft 126. Therefore, the motor 116 of the instrument drive system 110 may cause the shaft 126 of the medical instrument 120 to rotate. This allows the instrument drive system 110 to drive the medical instrument 120 without being mechanically coupled to the medical instrument 120. The motor 116 may cause the shaft 126 of the medical instrument 120 to rotate in either a clockwise direction or a counterclockwise direction around the longitudinal axis A of the rotor 125.

In some examples, the axis of rotation of the driving rotor in the motor 116 may be axially misaligned from the axis of rotation A of the driven rotor 125. For example, the misalignment may be caused by manufacturing tolerances of the housing 112 and the housing 122 (e.g., manufacturing tolerances of the drive axis of the motor 116 and the drive axis of the rotor 125) and/or manufacturing tolerances between the drive axis of the motor 116 or the drive axis of the rotor 125 to the other components of the instrument 120 and/or the instrument drive system 110. The magnetic coupling members 130, 140 may accommodate such misalignment and may still allow for the driving magnet 132 to rotate the driven magnet 142. This allows the instrument drive system 110 to drive the medical instrument 120 even when the axis of rotation of the driving rotor in the motor 116 is axially misaligned from the axis of rotation A of the driven rotor 125. For example, the gap 180 between the housing 112 of the instrument drive system 110 and the housing 122 of the medical instrument 120 may provide some clearance for the magnetic coupling members 130, 140 to be nested as shown in FIGS. 2A and 2B even if the axis of rotation of the driving rotor in the motor 116 is misaligned from the axis of rotation of the driven rotor 125.

FIGS. 3A and 3B illustrate a top view of the magnetic coupling member 130 and the magnetic coupling member 140. FIGS. 3A and 3B also show a rotational axis R of the magnetic coupling members 130, 140. In some examples, the rotational axis R is aligned with the longitudinal axis A of the rotor 125, as shown in FIG. 3A. In some examples, the rotational axis R is parallel with the longitudinal axis A but may be slightly offset from the longitudinal axis A, as shown in FIG. 3B. As discussed above, the magnet array 132 may include one or more individual magnets, such as the magnets 132A, 132B, 132C, and 132D. Each of the magnets 132A-D may be coupled to the housing 134 using any one or more of the coupling methods discussed above with respect to FIGS. 2A and 2B. While FIG. 3A shows the magnet array 132 including sixteen (16) magnets, the magnet array 132 may include any number of magnets. In some examples, the magnet array 132 includes an even number of magnets. In some examples, the magnet array 132 includes an odd number of magnets. In some examples, the magnet array 132 includes one magnet.

As shown in FIG. 3A, a polarity of each of the magnets 132A-D is pointed in a different direction. For example, the polarity 200A of the magnet 132A is pointed away from the rotational axis R of the magnetic coupling member 130. The pointing direction of the polarity 200A indicates which direction “North” is pointing for the magnet 132A. The pointing direction of the polarity 200B indicates which direction “North” is pointing for the magnet 132B. For example, the polarity 200B is pointed toward the rotational axis R of the magnetic coupling member 130. The pointing direction of the polarity 200C indicates which direction “North” is pointing for the magnet 132C. For example, the polarity 200C is pointed circumferentially clockwise along the circumference of the magnetic coupling member 130. The pointing direction of the polarity 200D indicates which direction “North” is pointing for the magnet 132D. For example, the polarity 200D is pointed circumferentially counterclockwise along the circumference of the magnetic coupling member 130. In some examples, one or both of the polarities 200C, 200D may be generally perpendicular (e.g., generally 90°) to one or both of the polarities 200A, 200B.

As further shown in FIG. 3A, the magnet array 132 may include more magnets in addition to the magnets 132A-D. In some examples, all of the magnets in the magnet array 132 may be arranged in a repeating pattern of polarity. For example, the polarity patterns of the magnets 132A-D may be repeated around the magnet array 132. While one particular polarity pattern is shown in FIG. 3A, any other polarity pattern may be used in the magnet array 132. In some examples, the magnet array 132 only includes magnets with polarities that point radially outward or inward, such as magnets 132A, 132B. This type of arrangement is shown in the magnet array 142 of the magnetic coupling member 140.

As discussed above, the magnet array 142 may include one or more individual magnets, such as the magnets 142A, 142B. Each of the magnets 142A, 142B may be coupled to the inner shaft 144 using any one or more of the coupling methods discussed above with respect to FIGS. 2A and 2B. While FIG. 3A shows the magnet array 142 including eight (8) magnets, the magnet array 142 may include any number of magnets. In some examples, the magnet array 142 includes an even number of magnets. In some examples, the magnet array 142 includes an odd number of magnets. In some examples, the magnet array 142 includes one magnet. In one example, the magnet array 132 and the magnet array 142 include the same number of magnetics, such as a single magnet each or the same even number of magnets.

As shown in FIG. 3A, a polarity of each of the magnets 142A, 142B is pointed in a different direction. For example, the polarity 205A of the magnet 142A is pointed away from the rotational axis R of the magnetic coupling member 140. The pointing direction of the polarity 205A indicates which direction “North” is pointing for the magnet 142A. The pointing direction of the polarity 205B indicates which direction “North” is pointing for the magnet 142B. For example, the polarity 205B is pointed toward the rotational axis R of the magnetic coupling member 140. As further shown in FIG. 3A, the magnet array 142 may include more magnets in addition to the magnets 142A, 142B. In some examples, all of the magnets in the magnet array 142 may be arranged in a repeating pattern of polarity. For example, the polarity patterns of the magnets 142A, 142B may be repeated around the magnet array 142. While one particular polarity pattern is shown in FIG. 3A, any other polarity pattern may be used in the magnet array 142.

FIG. 3C illustrates a top view of the magnetic coupling member 130 and the magnetic coupling member 140 along with a magnetic field 210. The housing 134 of the magnetic coupling member 130 includes an exterior surface 134A, an interior surface 134B, and a wall 134C extending between the exterior surface 134A and the interior surface 134B. The magnetic field 210 is illustrated by magnetic flux lines that show the magnetic interaction between the magnetic coupling member 130 and the magnetic coupling member 140.

The magnets of the magnet arrays 132, 142 may be arranged to reduce the amount of magnetic flux that extends beyond an outer diameter (e.g., beyond the exterior surface 134A) of the housing 134 of the magnetic coupling member 130. In some examples, the magnets of the magnet arrays 132, 142 may be arranged in a manner to keep the magnetic flux within the outer diameter of the housing 134. For example, as discussed above, the magnets 132C, 132D have polarities 200C, 200D that are pointed circumferentially along the circumference of the magnetic coupling member 130. These magnets help keep the magnetic flux closer to the exterior surface 134A of the housing 134. In some examples, these magnets keep the magnetic flux from extending beyond the exterior surface 134A. As shown in FIG. 3C, magnetic flux lines 220 of the magnetic field 210 are maintained within the outer diameter of the housing 134. Magnetic flux lines 225 of the magnetic field 210 extend beyond the outer diameter of the housing 134. The size, shape, orientation, and/or arrangement of one or more of the magnets in the magnet array 132 and/or the magnet array 142 may be adjusted to adjust how far the magnetic flux lines 225 extend beyond the outer diameter of the housing 134. In some examples, the size, shape, orientation, and/or arrangement of one or more of the magnets in the magnet array 132 and/or the magnet array 142 may be adjusted to keep the magnetic flux lines 225 within the outer diameter of the housing 134.

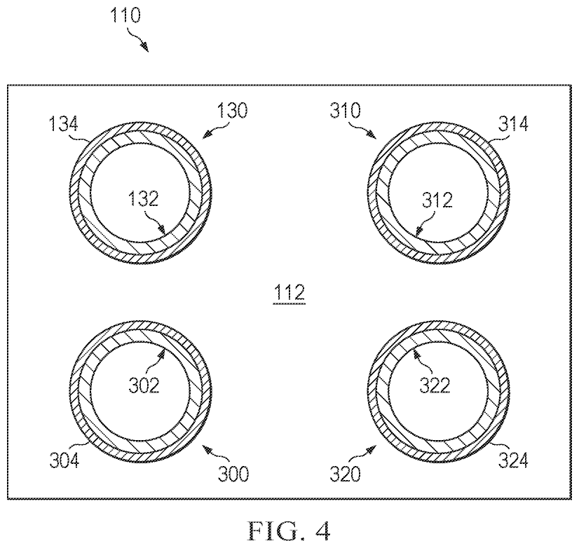

FIG. 4 illustrates a schematic top view of the housing 112 of the instrument drive system 110. In some examples, as shown in FIG. 4, the instrument drive system 110 may include more than one magnetic coupling member. For example, the instrument drive system 110 may include four magnetic coupling members 130, 300, 310, 320. Any other number of magnetic coupling members may be included in the instrument drive system 110, such as two magnetic coupling members, three magnetic coupling members, five magnetic coupling members, or any other number of magnetic coupling members. In the example shown in FIG. 4, the housing 112 of the instrument drive system 110 includes the housing 134 for the magnetic coupling member 130. The housing 112 may also include housings 304, 314, and 324, which respectively house three additional magnet arrays 302, 312, 322 of the magnetic coupling members 300, 310, 320. The details discussed above with respect to the magnetic coupling member 130 similarly apply to the magnetic coupling members 300, 310, 320.

As discussed above with respect to FIG. 3C, maintaining the magnetic flux within the boundaries of the housing 134 of the magnetic coupling member 130 may contain the magnetic field and the affects thereof within the outer boundary of the housing 134. When the instrument drive system 110 includes more than one magnetic coupling member, it is beneficial to reduce the effects of one magnetic coupling member on another magnetic coupling member. This may prevent the magnetic field of one magnetic coupling member from interfering with the magnetic field of another magnetic coupling member. Therefore, multiple magnetic coupling members with their own individual magnet arrays may be operated independently without affecting the performance of the remaining magnetic coupling members. For example, by maintaining the magnetic flux of the magnetic field 210 (FIG. 3C) within the boundaries of the housing 134, the effects of the magnetic field 210 on one or more of the magnetic coupling members 300, 310, 320 may be reduced and in some examples entirely removed.

The magnetic coupling members 130, 300, 310, 320 may be arranged to provide the least interference between the individual magnetic coupling members 130, 300, 310, 320. In some examples, the housings 134, 304, 314, 324 are spaced apart by at least a ¾ in gap. Any other spacing between the housings 134, 304, 314, 324 may be used. In some examples, the housings 134, 304, 314, 324 are equally spaced apart from each other. Alternatively, the spacings between the housings 134, 304, 314, 324 may be different. For example, the housing 134 may be spaced from the housing 304 by a first distance (e.g., ¾ in) and may be spaced from the housing 314 by a second distance (e.g., ⅞ in). Any other alternative spacings may be used. The spacings between the housings 134, 304, 314, 324 may be adjusted to minimize the interference between the individual magnetic coupling members 130, 300, 310, 320.

In the examples discussed above, the driven magnet 142 and the driving magnet 132 are in a nested configuration, with the driven magnet 142 nested concentrically inside the driving magnet 132. In other examples, the driven magnet 142 and the driving magnet 132 may be stacked on top of each other in an axial direction along the rotational axis R of the driving magnet 132. For example, the magnets 132, 142 may be in a “face-to-face” configuration, where the gap (e.g., an air gap) between the magnets 132, 142 extends in the axial direction.

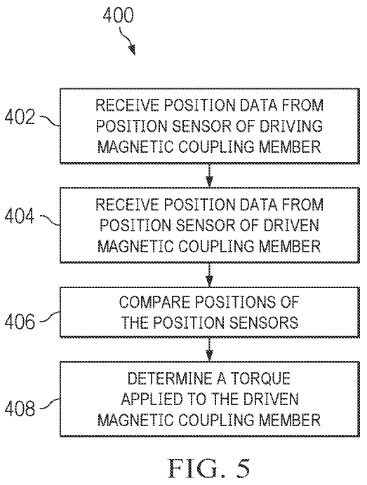

FIG. 5 is a flowchart illustrating a method 400 of determining a torque applied to a driven magnetic coupling member (e.g., the magnetic coupling member 140). The method 400 is illustrated as a set of operations or processes 402 through 408. The processes illustrated in FIG. 5 may be performed in a different order than the order shown in FIG. 5, and one or more of the illustrated processes might not be performed in some embodiments of the method 400. Additionally, one or more processes that are not expressly illustrated in FIG. 5 may be included before, after, in between, or as part of the illustrated processes.

A magnetic coupling, such as the magnetic coupling between the magnetic coupling member 130 and the magnetic coupling member 140, acts as a torsion spring. When the magnetic coupling is rotated, the rotational position of the magnetic coupling members that are magnetically coupled via the magnetic coupling changes. The rotational position of each magnetic coupling member, which may be a driving magnetic coupling member and a driven magnetic coupling member, may be independently measured. By measuring the difference between the rotational positions of the driving magnetic coupling and the driven magnetic coupling member, the torque provided by the magnetic coupling may be determined.

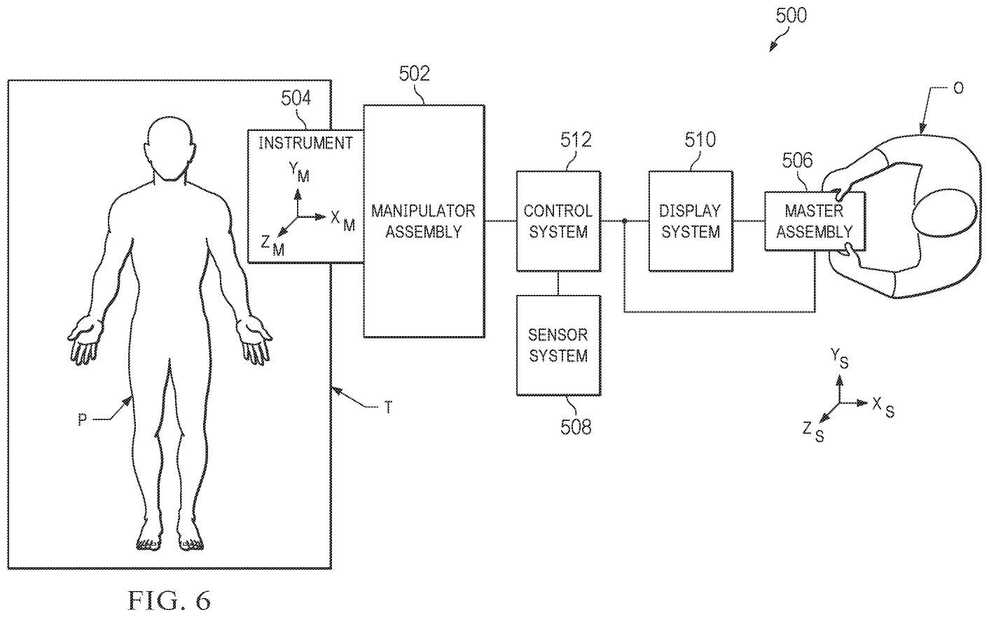

At a process 402, position data from a position sensor of the driving magnetic coupling member (e.g., the magnetic coupling member 130) is received. In some examples, a position sensor is coupled to the driving magnetic coupling member 130. For example, the position sensor may be coupled to the magnet 132 and/or to the housing 134. Position data from the position sensor may be received by a control system (e.g., the control system 512 of FIG. 6). The position data may be received by one or more processors and/or one or more memories of the control system 512. The position data may indicate the rotational position of the driving magnetic coupling member 130 about the rotational axis R of the driving magnetic coupling member 130. The position data may therefore indicate how many degrees the driving magnetic coupling member 130 has rotated from a 0° starting position. The starting position may be any position about the rotational axis R.

At a process 404, position data from a position sensor of the driven magnetic coupling member (e.g., the magnetic coupling member 140) is received. In some examples, a position sensor is coupled to the driven magnetic coupling member 140. For example, the position sensor may be coupled to the magnet 142 and/or to the inner shaft 144. Position data from the position sensor may be received by the control system 512 of FIG. 6. The position data may be received by one or more processors and/or one or more memories of the control system 512. The position data may indicate the rotational position of the driven magnetic coupling member 140 about the rotational axis R of the driven magnetic coupling member 140. The position data may therefore indicate how many degrees the driven magnetic coupling member 140 has rotated from a 0° starting position. The starting position may be any position about the rotational axis R. In some examples, the starting position for the position sensor coupled to the driven magnetic coupling member 140 is the same starting position as the starting position for the position sensor coupled to the driving magnetic coupling member 130.

At a process 406, the positions of the position sensor coupled to the driving magnetic coupling member 130 and the position sensor coupled to the driven magnetic coupling member 140 are compared. The relative position between the two position sensors may be determined. In some examples, the driving magnetic coupling member 130 remains stationary and the driven magnetic coupling member 140 is rotated relative to the driving magnetic coupling member 130. In such examples, the position sensor coupled to the driven magnetic coupling member 140 is rotated away from the position sensor coupled to the driving magnetic coupling member 130.

At a process 408, a torque applied to the driven magnetic coupling member 140 is determined. The torque may be determined based on the comparison between the positions of the position sensor coupled to the driving magnetic coupling member 130 and the position sensor coupled to the driven magnetic coupling member 140. For example, when the two position sensors are rotationally aligned, the torque applied to the driven magnetic coupling member 140 is zero. When the magnet coupling members 130, 140 are wound, e.g., when the driven magnetic coupling member 140 is rotated with respect to the driving magnetic coupling member 130, the torque applied to the driven magnetic coupling member 140 increases. The angle between the two position sensors increases as the torque applied to the driven magnetic coupling member 140 increases. As such, the torque applied to the driven magnetic coupling member 140 is determined based on the angle between the two position sensors. In some examples, when the difference between the position sensors coupled to the driven magnetic coupling member 140 and the driving magnetic coupling member 130 reaches a threshold value, the torque applied to the driven magnetic coupling member 140 is at a maximum torque, which may be a threshold torque. The threshold value may correspond to an orientation where a magnet (e.g., the magnet 142A) in the magnet array 142 of the driven magnetic coupling member 140 would be attracted to the next magnet over (e.g., the magnet 132B) in the magnet array 132 of the driving magnetic coupling member 130 if the driven magnetic coupling member 140 was rotated further. The determined torque measurement may allow the control system 512 to know how much torque is being provided to the medical instrument 120 through the instrument drive system 110.

In some examples, the components discussed above may be part of a robotic-assisted system as described in further detail below. The robotic-assisted system may be suitable for use in, for example, surgical, robotic-assisted surgical, diagnostic, therapeutic, or biopsy procedures. While some examples are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems and general robotic, general robotic-assisted, or robotic medical systems.

As shown in FIG. 6, a medical system 500 generally includes a manipulator assembly 502 for operating a medical instrument 504 (e.g., the medical instrument 120) in performing various procedures on a patient P positioned on a table T. The manipulator assembly 502 may be robotic-assisted, non-robotic-assisted, or a hybrid robotic-assisted and non-robotic-assisted assembly with select degrees of freedom of motion that may be motorized and/or robotic-assisted and select degrees of freedom of motion that may be non-motorized and/or non-robotic-assisted. The medical system 500 may further include a master assembly 506, which generally includes one or more control devices for controlling manipulator assembly 502. Manipulator assembly 502 supports medical instrument 504 and may optionally include a plurality of actuators or motors that drive inputs on medical instrument 504 in response to commands from a control system 512. The actuators may optionally include drive systems that when coupled to medical instrument 504 may advance medical instrument 504 into a naturally or surgically created anatomic orifice.

Medical system 500 also includes a display system 510 for displaying an image or representation of the surgical site and medical instrument 504 generated by sub-systems of sensor system 508. Display system 510 and master assembly 506 may be oriented so operator O can control medical instrument 504 and master assembly 506 with the perception of telepresence. Additional information regarding the medical system 500 and the medical instrument 504 may be found in International Application Publication No. WO 2018/195216, filed on Apr. 18, 2018, entitled “Graphical User Interface for Monitoring an Image-Guided Procedure,” which is incorporated by reference herein in its entirety.

In some examples, medical instrument 504 may include components of an imaging system (discussed in more detail below), which may include an imaging scope assembly or imaging instrument that records a concurrent or real-time image of a surgical site and provides the image to the operator or operator O through one or more displays of medical system 500, such as one or more displays of display system 510. The concurrent image may be, for example, a two or three-dimensional image captured by an imaging instrument positioned within the surgical site. In some examples, the imaging system includes endoscopic imaging instrument components that may be integrally or removably coupled to medical instrument 504. However, in some examples, a separate endoscope, attached to a separate manipulator assembly may be used with medical instrument 504 to image the surgical site. In some examples, as described in detail below, the imaging instrument alone or in combination with other components of the medical instrument 504 may include one or more mechanisms for cleaning one or more lenses of the imaging instrument when the one or more lenses become partially and/or fully obscured by fluids and/or other materials encountered by the distal end of the imaging instrument. In some examples, the one or more cleaning mechanisms may optionally include an air and/or other gas delivery system that is usable to emit a puff of air and/or other gasses to blow the one or more lenses clean. Examples of the one or more cleaning mechanisms are discussed in more detail in International Application Publication No. WO/2016/025465, filed on Aug. 11, 2016, entitled “Systems and Methods for Cleaning an Endoscopic Instrument”; U.S. patent application Ser. No. 15/508,923, filed on Mar. 5, 2017, entitled “Devices, Systems, and Methods Using Mating Catheter Tips and Tools”; and U.S. patent application Ser. No. 15/503,589, filed Feb. 13, 2017, entitled “Systems and Methods for Cleaning an Endoscopic Instrument,” each of which is incorporated by reference herein in its entirety. The imaging system may be implemented as hardware, firmware, software or a combination thereof which interact with or are otherwise executed by one or more computer processors, which may include the processors of the control system 512.

Control system 512 includes at least one memory and at least one computer processor (not shown) for effecting control between medical instrument 504, master assembly 506, sensor system 508, and display system 510. Control system 512 also includes programmed instructions (e.g., a non-transitory machine-readable medium storing the instructions) to implement some or all of the methods described in accordance with aspects disclosed herein, including instructions for providing information to display system 510.

FIG. 7 is a simplified diagram of a medical instrument system 600 according to some examples. Medical instrument system 600 includes a flexible elongate device 602, such as a flexible catheter (e.g., the medical instrument 120), coupled to a drive unit 604 (e.g., the instrument drive system 110). Elongate device 602 includes a flexible body 616 having proximal end 617 and distal end or tip portion 618. Medical instrument system 600 further includes a tracking system 630 for determining the position, orientation, speed, velocity, pose, and/or shape of distal end 618 and/or of one or more segments 624 along flexible body 616 using one or more sensors and/or imaging devices as described in further detail below.

Tracking system 630 may optionally track distal end 618 and/or one or more of the segments 624 using a shape sensor 622. Shape sensor 622 may optionally include an optical fiber aligned with flexible body 616 (e.g., provided within an interior channel (not shown) or mounted externally). The optical fiber of shape sensor 622 forms a fiber optic bend sensor for determining the shape of flexible body 616. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. patent application Ser. No. 11/180,389, filed on Jul. 13, 2005, entitled “Fiber Optic Position and Shape Sensing Device and Method Relating Thereto”; U.S. patent application Ser. No. 12/047,056, filed on Jul. 16, 2004, entitled “Fiber-Optic Shape and Relative Position Sensing”; and U.S. Pat. No. 6,389,187, filed on Jun. 17, 1998, entitled “Optical Fibre Bend Sensor”, each of which is incorporated by reference herein in its entirety. Sensors in some examples may employ other suitable strain sensing techniques, such as Rayleigh scattering, Raman scattering, Brillouin scattering, and Fluorescence scattering. In some examples, the shape of the elongate device may be determined using other techniques. For example, a history of the distal end pose of flexible body 616 can be used to reconstruct the shape of flexible body 616 over the interval of time. In some examples, tracking system 630 may optionally and/or additionally track distal end 618 using a position sensor system 620. Position sensor system 620 may be a component of an EM sensor system with position sensor system 620 including one or more conductive coils that may be subjected to an externally generated electromagnetic field. Each coil of the EM sensor system then produces an induced electrical signal having characteristics that depend on the position and orientation of the coil relative to the externally generated electromagnetic field. In some examples, position sensor system 620 may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point. Further description of a position sensor system is provided in U.S. Pat. No. 6,380,732, filed on Aug. 11, 1999, entitled “Six-Degree of Freedom Tracking System Having a Passive Transponder on the Object Being Tracked”, which is incorporated by reference herein in its entirety.

Flexible body 616 includes a channel 621 sized and shaped to receive a medical instrument 626. Further description of a medical instrument received by a flexible body is provided in U.S. Provisional Patent Application No. 63/077,059, filed on Sep. 11, 2020, entitled “Systems for Coupling and Storing an Imaging Instrument”, which is incorporated by reference herein in its entirety.

Flexible body 616 may also house cables, linkages, or other steering controls (not shown) that extend between drive unit 604 and distal end 618 to controllably bend distal end 618 as shown, for example, by broken dashed line depictions 619 of distal end 618. In some examples, at least four cables are used to provide independent “up-down” steering to control a pitch of distal end 618 and “left-right” steering to control a yaw of distal end 618. Steerable elongate devices are described in detail in U.S. patent application Ser. No. 13/274,208, filed on Oct. 14, 2011, entitled “Catheter with Removable Vision Probe”, which is incorporated by reference herein in its entirety.

The information from tracking system 630 may be sent to a navigation system 632 where it is combined with information from image processing system 631 and/or the preoperatively obtained models to provide the operator with real-time position information. In some examples, the real-time position information may be displayed on display system 510 of FIG. 6 for use in the control of medical instrument system 600. In some examples, control system 512 of FIG. 6 may utilize the position information as feedback for positioning medical instrument system 600. Various systems for using fiber optic sensors to register and display a surgical instrument with surgical images are provided in U.S. patent application Ser. No. 13/107,562, filed on May 13, 2011, entitled “Medical System Providing Dynamic Registration of a Model of an Anatomic Structure for Image-Guided Surgery,” which is incorporated by reference herein in its entirety.

In some examples, medical instrument system 600 may be robotic-assisted within medical system 500 of FIG. 6. In some examples, manipulator assembly 502 of FIG. 6 may be replaced by direct operator control. In some examples, the direct operator control may include various handles and operator interfaces for hand-held operation of the instrument.

The singular forms “a”, “an”, and “the” are intended to include the plural forms as well, unless the context indicates otherwise. And the terms “comprises,” “comprising,” “includes,” “has,” and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components. Components described as coupled may be directly or indirectly communicatively coupled. The auxiliary verb “may” likewise implies that a feature, step, operation, element, or component is optional.

In the description, specific details have been set forth describing some examples. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the following description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions.

Any alterations and further modifications to the described devices, instruments, methods, and any further application of the principles of the present disclosure are fully contemplated as would normally occur to one skilled in the art to which the disclosure relates. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

The systems and methods described herein may be suited for navigation and treatment of anatomic tissues, via natural or surgically created connected passageways, in any of a variety of anatomic systems, including the lung, colon, the intestines, the kidneys and kidney calices, the brain, the heart, the circulatory system including vasculature, and/or the like. Although some of the examples described herein refer to surgical procedures or instruments, or medical procedures and medical instruments, the techniques disclosed apply to non-medical procedures and non-medical instruments. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

Further, although some of the examples presented in this disclosure discuss robotic-assisted systems or remotely operable systems, the techniques disclosed are also applicable to computer-assisted systems that are directly and manually moved by operators, in part or in whole.

Additionally, one or more elements in examples of this disclosure may be implemented in software to execute on a processor of a computer system such as a control processing system. When implemented in software, the elements of the examples of the present disclosure are essentially the code segments to perform the necessary tasks. The program or code segments can be stored in a processor readable storage medium (e.g., a non-transitory storage medium) or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In some examples, the control system may support wireless communication protocols such as Bluetooth, Infrared Data Association (IrDA), HomeRF, IEEE 802.11, Digital Enhanced Cordless Telecommunications (DECT), ultra-wideband (UWB), ZigBee, and Wireless Telemetry.

A computer is a machine that follows programmed instructions to perform mathematical or logical functions on input information to produce processed output information. A computer includes a logic unit that performs the mathematical or logical functions, and memory that stores the programmed instructions, the input information, and the output information. The term “computer” and similar terms, such as “processor” or “controller” or “control system”, are analogous.

Note that the processes and displays presented may not inherently be related to any particular computer or other apparatus, and various systems may be used with programs in accordance with the teachings herein. The required structure for a variety of the systems discussed above will appear as elements in the claims. In addition, the examples of the present disclosure are not described with reference to any particular programming language. It will be appreciated that a variety of programming languages may be used to implement the teachings of the present disclosure as described herein.

While certain example examples of the present disclosure have been described and shown in the accompanying drawings, it is to be understood that such examples are merely illustrative of and not restrictive to the broad disclosed concepts, and that the examples of the present disclosure not be limited to the specific constructions and arrangements shown and described, since various other modifications may occur to those ordinarily skilled in the art.

1. A system, comprising:
an instrument drive system, comprising:
a first housing;
a first magnetic coupling member within the first housing; and
a motor mechanically coupled to the first magnetic coupling member, the motor configured to rotate the first magnetic coupling member; and
a medical instrument, comprising:
a second housing;
a rotor within the second housing; and
a second magnetic coupling member within the second housing and mechanically coupled to the rotor, the second magnetic coupling member being spaced from and magnetically coupled to the first magnetic coupling member; and
wherein rotation of the first magnetic coupling member causes rotation of the second magnetic coupling member and the rotor.

2. The system of claim 1, wherein the first housing of the instrument drive system seals the first magnetic coupling member and the motor within the first housing, and wherein the second housing of the medical instrument seals the rotor and the second magnetic coupling member within the second housing.

3. The system of claim 1, wherein the second magnetic coupling member is within a protruding portion of the second housing of the medical instrument, wherein the protruding portion is located within a recessed portion formed by the first housing of the instrument drive system, and wherein the first magnetic coupling member is positioned around an interior surface of the recessed portion.

4. The system of claim 1, wherein:
the first magnetic coupling member includes an aperture; and
the second magnetic coupling member is located within the aperture when the first housing is mechanically coupled to the second housing.

5. The system of claim 1, wherein the first magnetic coupling member is axially spaced from the second magnetic coupling member in a direction parallel with an axis of rotation of the first magnetic coupling member.

6. The system of claim 1, wherein the first magnetic coupling member includes a first plurality of magnets rotationally spaced around an axis of rotation of the first magnetic coupling member.

7. The system of claim 6, wherein the first plurality of magnets includes:
a first subset of magnets with a polarity directed radially outward;
a second subset of magnets with a polarity directed radially inward; and
a third subset of magnets with a polarity directed generally perpendicularly to the polarity of the first and second subsets of magnets.

8. (canceled)

9. The system of claim 1, wherein the system further comprises an air gap between the first magnetic coupling member and the second magnetic coupling member, and wherein a sterile barrier extends within the air gap.

10. The system of claim 1, wherein the second magnetic coupling member includes a second plurality of magnets rotationally spaced around an axis of rotation of the second magnetic coupling member.

11. (canceled)

12. The system of claim 1, wherein the medical instrument further comprises:
a flexible elongate device including a pull wire, the pull wire coupled to the rotor; and
a pull wire pulley coupled to the pull wire,
wherein rotation of the rotor changes a tension of the pull wire to control articulation of the flexible elongate device.

13-15. (canceled)

16. The system of claim 1, further comprising:
a first position sensor coupled to the first magnetic coupling member;
a second position sensor coupled to the second magnetic coupling member; and
a control system configured to:
receive position data from the first and second position sensors indicating a position of the first position sensor relative to the second position sensor; and
based on the received position data, determine a torque applied to the second magnetic coupling member.

17. (canceled)

18. A medical instrument, comprising:
a rotor;
a magnetic coupling member mechanically coupled to the rotor, the magnetic coupling member being configured to rotate via a magnetic coupling; and
a flexible elongate device, wherein the magnetic coupling member causes rotation of the rotor, and rotation of the rotor causes movement of the flexible elongate device.

19. The medical instrument of claim 18, wherein the magnetic coupling member includes a plurality of magnets rotationally spaced around an axis of rotation of the magnetic coupling member.

20. The medical instrument of claim 19, wherein the plurality of magnets includes:
a first subset of magnets with a polarity directed radially outward;
a second subset of magnets with a polarity directed radially inward; and
a third subset of magnets with a polarity directed generally perpendicularly to the polarity of the first and second subsets of magnets.

21. (canceled)

22. (canceled)

23. The medical instrument of claim 18, further comprising a housing that seals the magnetic coupling member and the rotor within the housing.

24. A system for coupling a medical instrument to an instrument drive system, the system comprising:
a first magnetic coupling member positioned within a housing of the instrument drive system, the first magnetic coupling member including a first position sensor;
a second magnetic coupling member spaced from and magnetically coupled to the first magnetic coupling member, the second magnetic coupling member including a second position sensor, the second magnetic coupling member mechanically coupled to the medical instrument and positioned within a housing of the medical instrument, wherein rotation of the first magnetic coupling member causes corresponding rotation of the second magnetic coupling member, and wherein the rotation of the second magnetic coupling member causes corresponding rotation of a rotor of the medical instrument; and
a control system comprising:
a processor; and
a memory comprising machine readable instructions that, when executed by the processor, cause the control system to:
receive position data from the first and second position sensors indicating a position of the first position sensor relative to the second position sensor; and
based on the received position data, determine a torque applied to the second magnetic coupling member.

25. The system of claim 24, wherein the first magnetic coupling member includes a magnet array, and wherein the first position sensor is coupled to the magnet array.

26. The system of claim 24, wherein the first magnetic coupling member includes a housing, and wherein the first position sensor is coupled to the housing.

27. (canceled)

28. (canceled)

29. The system of claim 24, wherein the control system is further configured to compare the position data from the first position sensor with the position data from the second position sensor, and wherein determining the torque applied to the second magnetic coupling member is based on the comparison.

30. (canceled)

31. The system of claim 24, wherein the system is further configured to determine a torque applied to the medical instrument based on the torque applied to the second magnetic coupling member.

32. (canceled)
